(19) United States
(12) Patent Application Publication
(10) Pub. No.: US 2012/0016373 A1
(43) Pub. Date: Jan. 19, 2012

(54) DEVICE TO ASSIST WITH THE PLACEMENT OF SCREWS IN BONE TISSUE AND INSTRUMENT APPLYING SAID DEVICE, IN PARTICULAR FOR PERFORMING OSTEOSYNTHESIS OF BONE FRAGMENTS

(75) Inventor: Frédéric Impellizzeri, Salon de Provence (FR)

(73) Assignee: BIOTECH INTERNATIONAL (SOCIETE PAR ACTIONS SIMPLIF IEE), Salon de Provence (FR)

(21) Appl. No.: 13/146,145

(22) PCT Filed: Jan. 26, 2010
(86) PCT No.: PCT/FR2010/000063

§ 371 Date: Sep. 29, 2011

(30) Foreign Application Priority Data
Feb. 19, 2009 (FR) 09/00757

(51) Int. Cl. A61B 17/56

(52) U.S. Cl. 606/104

(57) ABSTRACT

A device to assist with the placement of screws in bone tissue, characterised in that said device includes an elongate hollow body comprising: a proximal portion enabling the attachment thereof to any rotating device, a distal portion provided with a milling cutter for making the recess for receiving the screw heads, a central portion provided with a longitudinal window with grading and, a drill spindle with a diameter enabling the axial sliding thereof into the bore of the hollow body and comprising, in the intermediate portion thereof, a marking means suitable for being positioned opposite the window of said body. The proximal portion of the body being arranged to enable either simultaneous rotation of the body and the spindle housed in the latter, or rotation of only the body.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns a device facilitating the placement of screws in bone tissue, in particular in orthopedic surgery. It also deals with the operational instruments making use of the device according to the invention.

2. Description of Related Art Including Information Disclosed Under 37 CFR 1.97 and 37 CFR 1.98.

The operative procedure known for placing screws in bone tissue, for instance to reduce a fracture through osteosynthesis and in particular for placing hollowed-out screws, is the following:

bore through the bone fragments by means of a generally smooth and pointed spindle, put in rotation by a motor equipped with a spindle-passing tip;

replace the spindle-passing tip with a fast interlocking tip of the type known under the name of “A.O. Synthes”, or with a universal three-jaw chuck;

use a hollow and graduated drill whose function is to prepare a seat for the screw and to measure the depth of the hole in order to determine the length of the screw to be used;

use a hollowed-out milling cutter to obtain a recessed hole for the head of the screw; and withdraw the milling cutter and place the screw.

Such an operation which requires the successive manipulation of several instruments can, depending on the fracture reduction cases to be treated, turn out to be a relatively long and complex one.

An instrument has been proposed, in the field of orthopedic surgery (WO-2008/036.309) that makes it possible to drill a guiding passage in a bone while measuring the depth of this passage during its execution in order to determine the length of the screws to be used.

This instrument consists of a non-cannulated drill which comprises a cutting portion and a shaft portion with a number of rings. The gauge also includes a drilling sleeve and a graduated scale with a female component and a male component with a front end and a rear end and a number of rings at the rear end.

A major disadvantage of such an instrument is that it cannot serve to facilitate and select the proper positioning of the osteosynthetic screws, such as compression screws because no advance check of the direction of said screws is possible, as this is done if the guide spindle is used, which means that their insertion may turn out to be defective.

Another major disadvantage of the instrument described in the WO-2008/036.309 document is that it does not noticeably reduce the time required for placing orthopedic screws, on account of the fact that the drilling and recess creating operations need to be done separately, by different instruments. The time that the patients are under anesthesia is therefore longer which constitutes a constraint, for the surgeon as well as for the patients.

Document US 2006/0184174 describes an adjustable drill for use in the implantation of orthopedic screws such as adjustable compression screws with a separate adjustable head. The adjustable drill comprises a main drill and a milling drill which surrounds the first drill. The milling drill is configured to slide selectively along the main drill in order to use it by selecting a drilling depth. The milling drill is associated with a stopping member which is configured to selectively lock the milling drill in one of the positions selected along the main drill in order to thereby establish a drilling depth. The depth of the pre-bore corresponds to the size of the screw selected by the surgeon.

This instrument allows the drilling of passages in a bony material proportional to the length of the screws the surgeon intends to use. Now, in the majority of fracture reduction cases, it is not possible to determine the length of the screws to be used before knowing the depth of the pre-bores performed in the bone fragments to be brought together again.

On the other hand, this instrument also has the previously mentioned disadvantage of not predicting the positioning of the screws.

In certain cases, practitioners prefer to forgo this type of operative procedure for the benefit of using self-boring screws and to save the operating time corresponding to the boring.

One aim of the present invention is to remedy these disadvantages by offering surgeons a reliable solution in the form of a more precise and faster placement of screws for the purpose of osteosynthesis.

### BRIEF SUMMARY OF THE INVENTION

According to the invention, this aim is achieved through a polyfunctional device capable of performing all the steps involved in positioning orthopedic screws in bone tissues, in particular to achieve osteosynthesis of bone fragments. This device comprises:

on the one hand, an elongated hollow body featuring:
a proximal portion permitting its fastening on a chuck and preferably on a spindle-pass or other rotational driving device,
a distal portion equipped with a milling cutter capable of producing a recess space for receiving the head of the screws,
a central portion equipped with a longitudinal readout viewport with a graduated scale and, on the other hand,
a perforating spindle with a diameter permitting its axial slide in the bore of the hollow body and featuring, in its intermediate portion, a marker that is intended to become visible in the readout viewport of said body;
the proximal portion of the body being fitted to enable either the simultaneous rotational drive of the body and the spindle housed inside it, or the drive of said body only.

Using the device according to the invention, the protocol for placing orthopedic screws is as follows: introduce the spindle inside the hollow body so that the distal portion of the spindle extends over the distal portion of said body; with the chuck of a rotational drive apparatus and preferably on a spindle-pass, clamp simultaneously the body and the spindle; bore a passage in the bone tissues through the intermediary of the spindle, under radiographic control; interrupt the rotation of the assembly when the passage made has obtained the desirable depth, i.e., when the distal end of the spindle has reached the opposite cortical wall, and loosen the drive chuck; slide the hollow body along the spindle until the distal end of the body constituted by the end of the milling cutter comes to rest against the upper cortical of the bone; on the readout viewport, read the length of the screw to be used; position and tighten the chuck of the drive device so it will drive only said hollow body in rotation; mill the upper cortical of the bone in order to create the recess for receiving the head of the screw; release the chuck from the driving device and withdraw the hollow body while leaving the spindle in position in the bone; place a self-tapping screw, featuring an axial bore and presenting the adequate length, around the spindle and slide it along on it so as to bring it into contact with the osseous material; and with a screwdriver also featuring an axial bore enabling its slide and its rotation along and around the spindle, perform the fastening of the screw in the osseous material.

The device according to the invention offers several interesting advantages. It permits in particular: a precise determination of the length of orthopedic screws to be used; a significantly facilitated precise positioning; utilization of a single instrument, and hence fewer sources of error; and less time for placing orthopedic screws and consequently a reduction of the time the patient is under anesthesia.

According to an advantageous method of execution, the extreme portion of the proximal part of the hollow body is provided with longitudinal apertures, per example with four evenly spaced longitudinal apertures. This arrangement permits fastening the body to the spindle and to ensure the simultaneous rotational drive of said body and said spindle.

According to a preferred embodiment, the extreme slotted portion of the proximal part of the body presents a diameter that is smaller than that of the adjoining non-slotted portion of said proximal part.

According to an advantageous embodiment, the hollow, cylindrical or approximately cylindrical body includes a flattened central part provided with the graduated scale viewport.

According to a preferred embodiment, the viewport is constituted by a rectangular aperture made in one of the faces of the flattened central part of the body, and is provided, on at least one of its edges, with a graduation.

This graduation is, for example, constituted, on one of the aperture's edges, by unitary grades and on the other side of said aperture by numerical markers that are multiples of five.

According to another advantageous embodiment, the two opposite faces of the flattened central portion of the body are provided with a graduated longitudinal aperture on one of the edges of said aperture, so that reading the depth of the hole might be possible no matter what the position of the instrument.

According to an advantageous embodiment, the spindle is equipped, in its intermediary portion at a predetermined distance from its active point, with a means of marking which may consist of a ring or a colored area, or preferably, by a ring or an area directly engraved in the material the spindle is made of.

### BRIEF DESCRIPTION OF THE DRAWINGS

The above aims, characteristics and advantages and still others will become clearer from the description below and the attached drawings in which:

FIG. 1 is a front view of of one example of embodiment of the device according to the invention.

FIG. 2 is a side view and in axial section of the hollow body.

FIGS. 3, 4 and 5 are views in radial section and at an enlarged scale along lines 3-3, 4-4 and 5-5 respectively of FIG. 2.

FIG. 6 is a perspective view showing the device shown with a capability of sliding on a piercing spindle.

FIGS. 7A and 7B illustrate two embodiments of the piercing spindle.

FIG. 8 illustrates the spindle in place in the hollow body during the bone piercing step of the bone screw protocol.

FIG. 9A is a view with partial section showing the spindle in place in the different bone fragments.

FIG. 9B represents the step of reading the depth of the hole in the procedure for putting bone screws in place.

FIG. 10 is a view illustrating the milling step of the procedure for putting bone screws in place.

FIG. 11 is a view representing a self-piercing screw in position on the spindle.

FIG. 12 is a view illustrating the placement of a self-piercing screw in the bone with a screwdriver.

FIG. 13 shows an orthopedic screw placed in the bone.

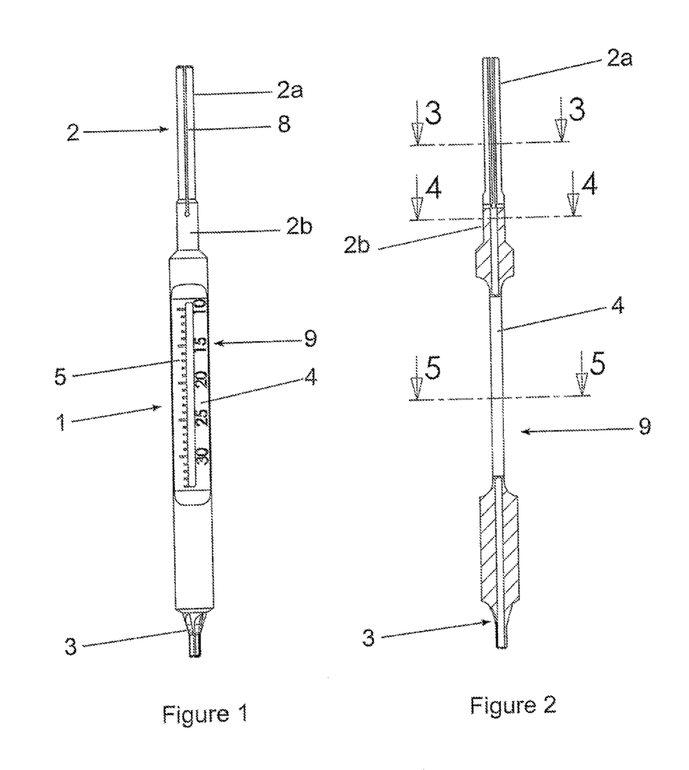

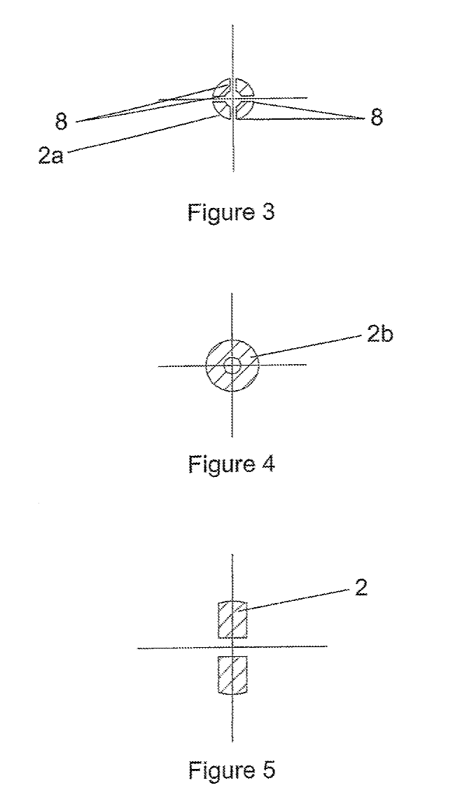

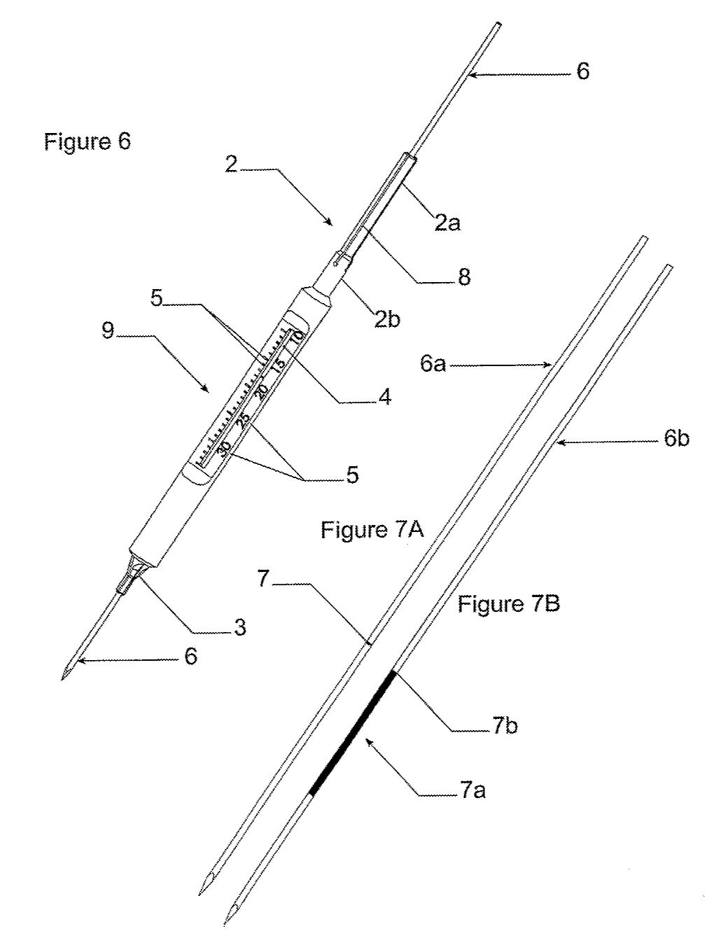

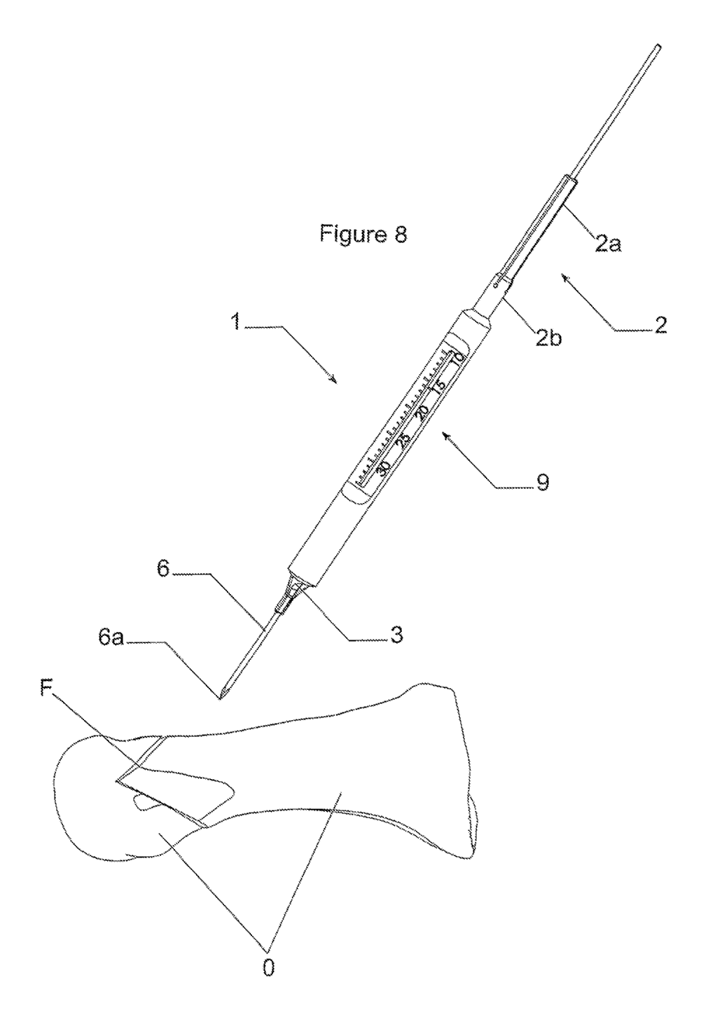

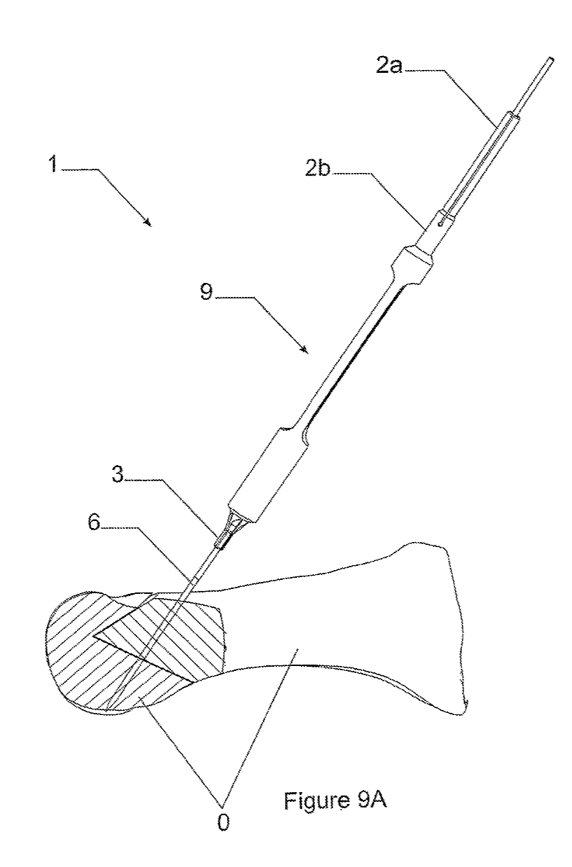

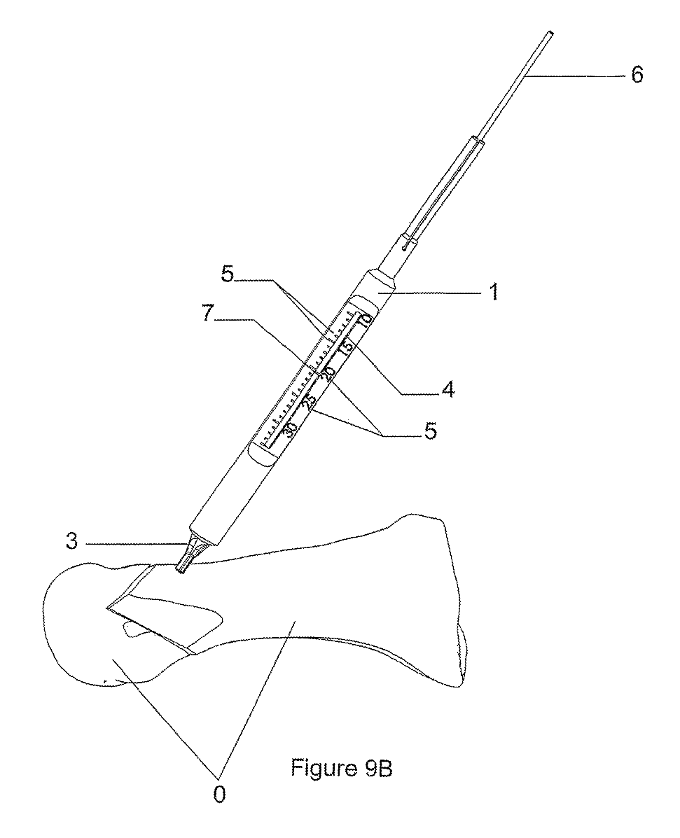

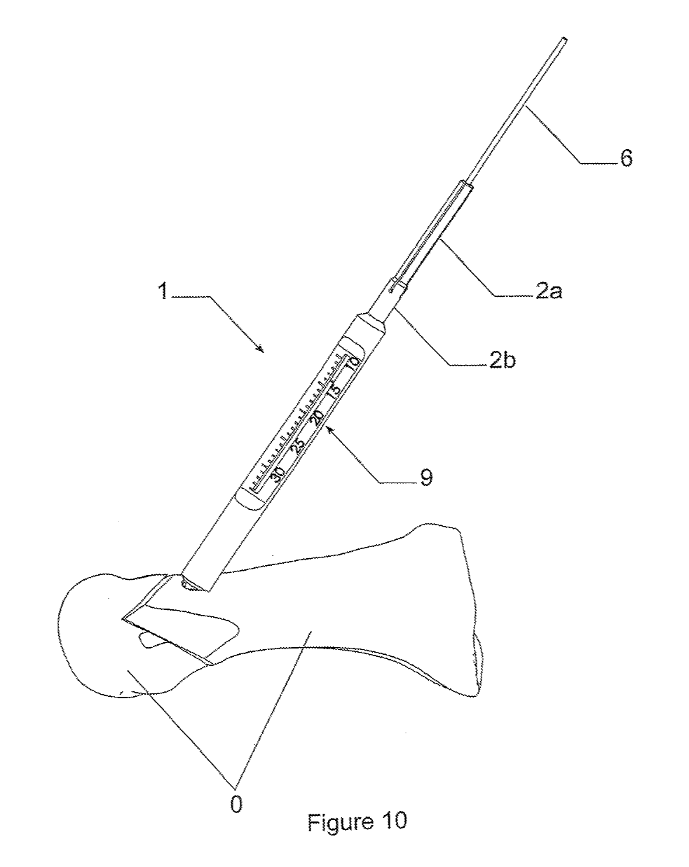

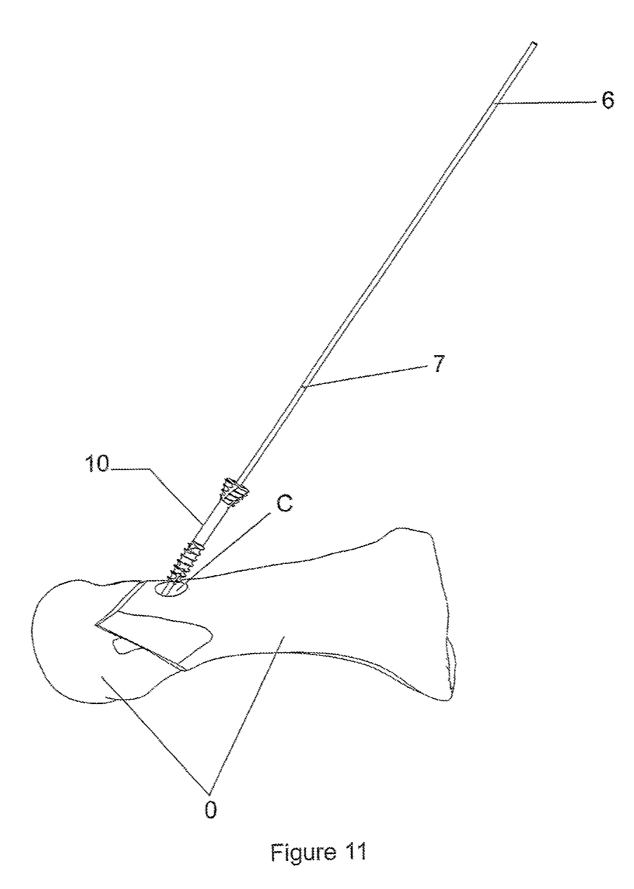

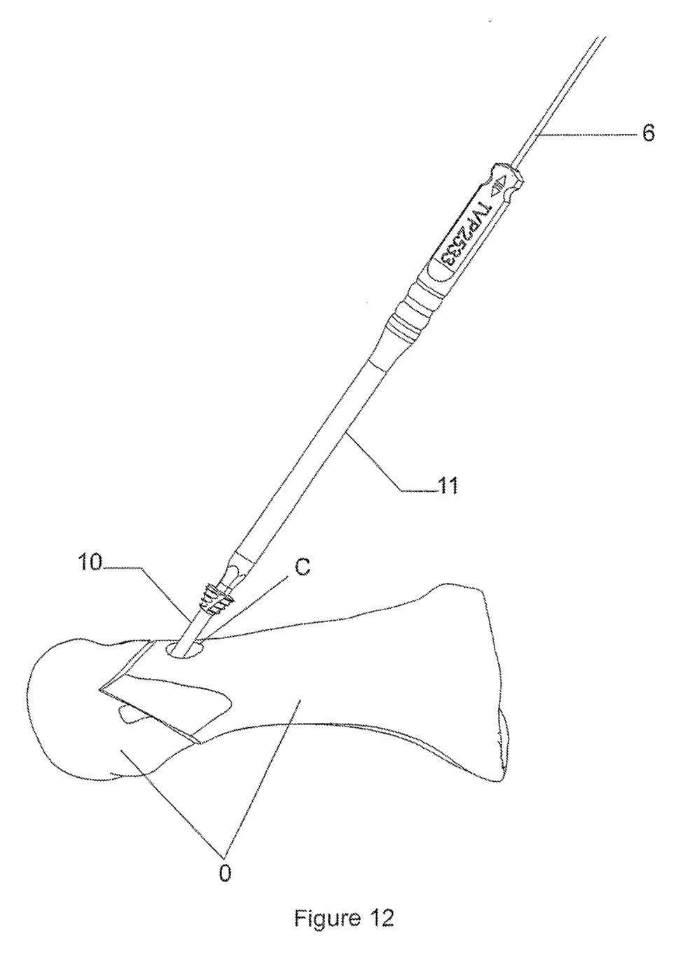

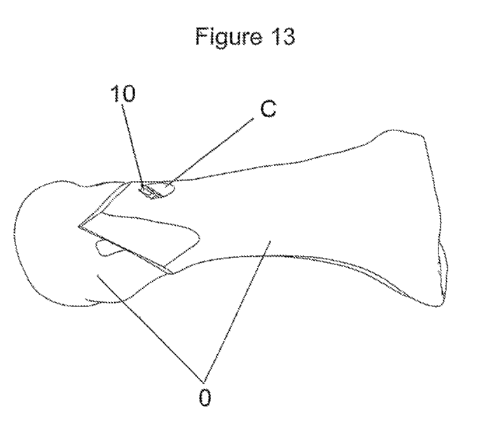

Reference is made to said drawings to describe an interesting although by no means limiting embodiment of the device for placing screws in bone tissue according to the invention.

### DETAILED DESCRIPTION OF THE INVENTION

The device according to the invention comprises primarily:

on the one hand, an elongated hollow body 1 featuring:
a proximal portion 2 enabling its fastening to a spindle-passing chuck or another rotational drive device,
a distal portion equipped with a formed cutter 3 capable of creating the recess for receiving a screw head,
a central portion provided with a longitudinal viewport 4 featuring a graduation 5 and, on the other hand,
   a spindle with trocar tip 6 presenting a diameter allowing its axial slide in the bore of the hollow body 1 and featuring, in its intermediary portion, a means of marking 7 capable of being shown in the viewport 4 of said body;
   the proximal portion 2 of the body 1 being fitted so that either the body 1 and the spindle 6 housed in the body may be driven simultaneously in rotation or so that only said body may be driven.

According to the example shown, the extreme portion 2a of the proximal part 2 of the hollow body 1 is provided with longitudinal apertures 8, for example four longitudinal evenly spaced apertures. This arrangement permits tightening the body 1 on the spindle 6 and providing the simultaneous rotational drive of said body and spindle.

According to the example shown, the proximal portion 2 of the hollow body 1 is also provided with a portion 2b for connecting said end portion 2a to the remaining part of the hollow body.

Advantageously, the lateral wall of the slit extreme portion 2a of the proximal portion of the body presents a diameter that is smaller than the diameter of the adjacent non-slit portion 2b of said proximal part. This arrangement makes it possible to feel a hard spot during the passage from one configuration to the other.

Likewise, the lateral wall of the non-slit portion 2b of a body 1 has a diameter smaller than the rest of said body.

According to the process of the invention, the hollow, cylindrical or approximately cylindrical body 1 includes a flattened central part 9 that is equipped with the graduated viewport.

According to the method of execution shown, the viewport 4 includes a rectangular longitudinal slot made in one of the faces of the flattened central part 9 of the body 1, and is equipped, on at least one of its edges, with a scale of 5.

This scale of 5 is, for example, constituted on one of the slot edges, by a graduation in millimeters, and on the other side of said slot, by numerical markers that are multiples of 5.

Preferably and advantageously, the two opposite faces of the flattened central portion 9 of the hollow body 1 are provided with a graduated longitudinal slot 4 on one of the edges of said slot, so that it is possible to read the depth of the hole no matter what the position of the instrument might be.

According to the example shown, the spindle 6 is provided, in its intermediary portion, at a predetermined distance from its active tip 6a, with a ring-shaped mark 7 which may include a colored ring, or preferably, by a ring directly engraved in the spindle material so as to be visible across the viewport 4. The surgeon can thus read the position of the ring on the corresponding graduations and determine the length of the orthopedic screw to be used.

Preferably and advantageously, the spindle 6 is provided, in its intermediary portion, at a predetermined distance from its active tip 6a, with an annular area 7a which may consist of a colored covering, or preferably be directly engraved in the spindle material so as to be visible across the viewport 4. The surgeon can thus read the position of the transition 7b between the engraved area and the neutral area on the corresponding graduations and determine the length of the orthopedic screw to be used.

The spindle 6 preferably presents a smooth surface over its entire length and its distal drilling end 6a is pointed.

The body 1 may be executed in any material of medical grade presenting the necessary hardness, such as for example stainless steel, titanium, etc.

On the other hand, the spindle 6 may be executed in any bio-compatible material presenting the necessary hardness, such as for example stainless steel, titanium, etc.

The instrument according to the invention includes hollow orthopedic screws, preferably compression screws presenting an axial bore of a diameter enabling easy sliding and rotation but without excessive play of said screws along and around the spindle 6.

On the other hand, this instrument includes also a screwdriver 11 that is used to place the orthopedic screws 10 in the pre-bored holes made by the device according to the inventions and is also provided with an axial bore of a diameter enabling easy sliding and rotation but without excessive play of said screwdriver along and around the spindle 6.

According to the operative protocol for placing bone screws using the device according to the invention, it is necessary to introduce the spindle 6 inside the hollow body 1 so that the distal part of the spindle extends considerably over the distal part of said body and simultaneously said body and said spindle are tightened by means of the chuck of a rotational driving apparatus.

This placement is monitored on a screen, for example an x-ray screen, on which the surgeon can visualize the progression of the spindle 6 in the bone tissues until the active tip 6a has gone through the bone fragments O, the fracture F, and until it comes into contact with the interior surface of the lower cortical of the bone.

When the spindle 6 reaches the desired position, the rotation of the body 1-spindle 6 assembly is stopped and the chuck of the rotational driving apparatus is loosened up.

The body 1 is then free to slide along the spindle 6 until the end of the body, comprising the milling cutter 3, comes to rest against the upper cortical of the bone O.

The surgeon can then read on the viewport 4, the depth of the pre-bore thus created and determine with absolute accuracy the length of the screw to be used.

The practitioner will then tighten only the body 1 by positioning the chuck of the rotational drive apparatus at the level of the portion 2b of the proximal part 2 of the body 1. The motor drives in rotation only said body and its milling cutter of which will create the recess C for receiving the head of the screw 10.

When the milling is done, the body 1 is withdrawn to leave only the spindle 6 in position in the bone.

The surgeon then places a self-tapping screw 10, featuring an axial bore 10a and presenting the appropriate length around the spindle 6 and makes it slide on the spindle in order to bring it into contact with the bone tissue.

By means of a screwdriver 11 featuring also an axial bore 11a permitting its slide and its rotation, along and around the spindle 6, the practitioner performs the fastening of the orthopedic screw 10 in the bone tissue. The screw is now in place in the bone tissue; the screwdriver 11 and spindle 6 are withdrawn.

1. Device to facilitate the placement of screws in bone tissue, in particular for achieving osteosynthesis of bone fragments characterized in that it comprises:
one the one hand, an elongated hollow body featuring:
a proximal part enabling its fastening to a chuck or other rotational drive device,
a distal part provided with a milling cutter for creating a recess area for receiving the head of the screws,
a central part provided with a longitudinal viewport provided with a graduation and, on the other hand,
a piercing spindle presenting a diameter permitting its axial slide in the bore of the hollow body and featuring, in its intermediary part, a means of marking that can be made visible in the viewport of said body;
the proximal part of the body being fitted to permit either the simultaneous rotational drive of the body and the spindle inside the body, or the drive of said body only.

2. Device according to claim 1, characterized in that the extreme portion of the proximal part of the hollow body is provided with longitudinal slots making it possible to tighten it in concentric fashion on the spindle.

3. Device according to claim 2, characterized in that the proximal part of the hollow body is constituted by two fastening portions, namely an end portion provided with longitudinal slots and enabling the simultaneous rotational drive of the body and the spindle inside the body, and a portion for connecting said end portion to the remaining part of the hollow body, permitting the rotational drive of the latter only.

4. Device according to claim 3, characterized in that the diameter of the side wall of the slotted extreme portion is smaller than the diameter of the side wall of the adjacent non-slotted portion of the proximal part of said hollow body.

5. Device according to claim 1, the hollow body of which presents a general cylindrical or approximately cylindrical shape, characterized in that said body presents a flattened central part of which at least one face is provided with a graduated viewport.

6. Device according to claim 5, characterized in that the two opposite faces of the flattened central part of the hollow body are provided with a graduated viewport.

7. Device according to claim 1, characterized in that the viewport or each viewport is constituted by a longitudinal aperture made in the central part of the hollow body and provided with a graduation on at least one of its edges.

8. Devise according to claim 7, characterized in that said graduation comprises, on the one hand, a graduation in millimeters distributed along one of the edges of the viewport and, on the other hand, numerical markers that are multiples of 5, distributed along the opposite edge of said viewport.

9. Device according to claim 1, characterized in that the spindle is equipped, in its intermediary portion, at a predetermined distance from its active tip with a ring-shaped mark which may comprise a colored ring or preferably by a ring directly engraved in the material constituting the spindle, or by an annular area which may be constituted by a colored covering, or preferably, be directly engraved in the material of the spindle.

10. Instrument to facilitate the placement of screws in bone tissues, in particular to achieve osteosynthesis of bone fragments according to claim 1, characterized in that the instrument also comprises compression screws or other orthopedic screws provided with an axial bore the diameter of which enables them to slide and rotate easily, but without excessive play, along and around the spindle.

11. Instrument according to claim 10, characterized in that it also includes a screwdriver provided with an axial bore the diameter of which permits its easy slide and rotation, but without excessive play, along and around the spindle.
